(19) United States
(12) Patent Application Publication
(10) Pub. No.: US 2026/0053969 A1
(43) Pub. Date: Feb. 26, 2026

(54) AROMA DIFFUSER

(71) Applicant: LONGWEI ZHANG, Dongguan (CN)

(72) Inventor: LONGWEI ZHANG, Dongguan (CN)

(21) Appl. No.: 18/888,256

(22) Filed: Sep. 18, 2024

(30) Foreign Application Priority Data
Aug. 21, 2024 (CN) 202422028900.9

(51) Int. Cl. A61L 9/14

(52) U.S. Cl. CPC A61L 9/14; A61L 2209/11; A61L 2209/133

(57) ABSTRACT

Disclosed is an aroma diffuser, including a housing and an atomizing device. The housing is provided with an object mounting site, an air outlet, and an atomizing mounting site. The atomizing device includes an atomizing shell, an atomizing sheet, and an atomizing elastic member. At least one atomizing hole is formed in the atomizing sheet, and the atomizing shell is provided with an atomizing snap-fit site. During assembly, the atomizing sheet, the atomizing elastic member, and the atomizing shell are assembled outside the housing to form the atomizing device. Then the entire atomizing device is inserted into the housing, and the atomizing snap-fit site is snap-fitted on the atomizing mounting site to achieve mounting and fixation of the atomizing device to the housing. A simple structure and the assembly outside the housing avoid positioning of the atomizing sheet and the atomizing elastic member with the housing.

### CROSS-REFERENCE TO RELATED APPLICATIONS

The application claims priority to Chinese Patent Application No. 202422028900.9, filed on Aug. 21, 2024 which is hereby incorporated by reference in its entirety.

### TECHNICAL FIELD

The present disclosure belongs to the field of aroma diffusers, and particularly relates to an aroma diffuser.

### BACKGROUND

A conventional aroma diffuser emits fragrance through evaporation of essential oil, which can optimize scent of air, improve ambient air, keep fresh the air in an entire environment, and create a comfortable living environment, thereby making a user feel pleasant. Aroma diffusion is capable of alleviating increasing life stress caused by a fast pace of modern life, and improving the quality of life, thereby becoming more and more popular among the public.

In the prior art, an aroma diffuser includes a housing, an atomizing sheet, and an amplitude adjusting device. The housing is provided with a mounting position for placing the atomizing sheet and the amplitude adjusting device. The atomizing sheet and the amplitude adjusting device are mounted in the housing. Through high-frequency vibration of the atomizing sheet, a mixture of essential oil and water is atomized and diffused into air, such that an entire space is filled with fragrance. However, the aroma diffuser of this structure has defects. The amplitude adjusting device and the atomizing sheet need to be positioned in the housing, such that when the housing structure is small, assembly time and costs are increased due to difficult and low-efficiency assembly and positioning of the amplitude adjusting device and the atomizing sheet in the housing.

Further, the conventional aroma diffuser is relatively single in functions, and is not suitable for some indoor scenarios of use.

### SUMMARY

### (1) Technical Problems to be Solved

In view of the defects in the prior art, an objective of the present disclosure is to provide an aroma diffuser to solve the technical problem of the prior art that assembly time and costs are increased due to difficult and low-efficiency assembly and positioning of an amplitude adjusting device and an atomizing sheet in a housing.

### (2) Technical Solutions

An aroma diffuser includes a housing and an atomizing device. The housing is provided with an atomizing mounting site, the atomizing device includes an atomizing shell, an atomizing sheet and an atomizing elastic member, at least one atomizing hole is formed in the atomizing sheet, and the atomizing shell is provided with an atomizing snap-fit site corresponding to the atomizing mounting site. The housing is provided with an object mounting site and an air outlet, the object mounting site can be configured for fixing an aroma bottle, and the atomizing device is arranged between the object mounting site and the air outlet. During assembly, the atomizing sheet, the atomizing elastic member, and the atomizing shell are assembled outside the housing to form the atomizing device. Then the entire atomizing device is inserted into the housing, and the atomizing snap-fit site is snap-fitted on the atomizing mounting site to achieve mounting and fixation of the atomizing device to the housing. A simple structure and the assembly outside the housing avoid positioning of the atomizing sheet and the atomizing elastic member with the housing, thereby effectively improving assembly efficiency and reducing the assembly time and costs. When in use, the aroma bottle is placed at the object mounting site, and the atomizing sheet corresponds to an opening of the aroma bottle. When the atomizing elastic member and the atomizing sheet vibrate at a high frequency at the opening of the aroma bottle, liquid in the aroma bottle, after passing through the atomizing hole, is mixed with a water mist formed by air and then discharged from the air outlet, to achieve uniform atomization and diffusion of diffuser liquid.

### (3) Beneficial Effects

Compared with the prior art, the present disclosure has the beneficial effects as follows:
1. The object mounting site can be configured for fixing an aroma bottle, and the atomizing device is arranged between the object mounting site and the air outlet. During assembly, the atomizing sheet, the atomizing elastic member, and the atomizing shell are assembled outside the housing to form the atomizing device. Then the entire atomizing device is inserted into the housing, and the atomizing snap-fit site is snap-fitted on the atomizing mounting site to achieve mounting and fixation of the atomizing device to the housing. A simple structure and the assembly outside the housing avoid positioning of the atomizing sheet and the atomizing elastic member with the housing, thereby effectively improving assembly efficiency and reducing the assembly time and costs.
2. In the present disclosure, a position of the atomizing sheet corresponds to an outflow hole of a bottleneck limiting block. The lower housing is provided with a control circuit device, and the atomizing sheet is electrically connected to the control circuit device. The atomizing sheet is provided with an atomizing site, and the atomizing hole is formed in the atomizing site. The atomizing site includes a concave surface and a convex surface, where the concave surface is arranged on a side of the outflow hole, and the convex surface is arranged on a side of the mist discharge hole. Under control of the control circuit device, high-frequency vibration caused by the atomizing sheet results in that essential oil in the aroma bottle is decomposed into nano-scale cold mist with a diameter of 0.1-5 microns at a position of the concave surface, and then the cold mist is diffused in surrounding air gradually through the convex surface, such that the air is filled with fragrance. Arrangement of the concave surface and the convex surface enhances the atomizing effect, broadens a dispersion range, and improves atomizing efficiency.

### BRIEF DESCRIPTION OF THE DRAWINGS

FIG. 1 is a schematic diagram of a three-dimensional structure of an aroma diffuser of the present disclosure.

FIG. 2 is a schematic diagram of a three-dimensional structure of an aroma bottle and an aroma diffuser fixed to each other according to the present disclosure.

FIG. 3 is an exploded view I of an aroma bottle and an aroma diffuser fixed to each other according to the present disclosure.

FIG. 4 is an exploded view II of an aroma bottle and an aroma diffuser fixed to each other according to the present disclosure.

FIG. 5 is a sectional view of an aroma bottle and an aroma diffuser fixed to each other according to the present disclosure.

FIG. 6 is an exploded view of an atomizing mounting site and an atomizing device of the present disclosure.

FIG. 7 is a schematic diagram of a three-dimensional structure of an atomizing device fixed at an atomizing mounting site according to the present disclosure.

FIG. 8 is a sectional view of two plugs in a horizontal state according to the present disclosure.

FIG. 9 is a sectional view of two plugs in a vertical state according to the present disclosure.

Reference numerals in the figures: 1. lower housing; 11. object mounting site; 111. mounting bracket; 112. avoidance channel; 113. positioning latch; 12. atomizing mounting site; 121. vertical mounting plate; 122. horizontal mounting plate; 123. bottleneck limiting block; 124. outflow hole; 125. recess; 13. lower rotating mounting arc groove; 2. upper housing; 21. air outlet; 22. upper rotating mounting arc groove; 23. decorative board; 3. atomizing device; 31. atomizing shell; 311. atomizing chamber; 312. mist discharge hole; 313. stop block; 32. atomizing sheet; 321. atomizing site; 322. concave surface; 323. convex surface; 33. atomizing elastic member; 34. atomizing hole; 35. snap latch; 4. control circuit device; 41. control switch; 5. aroma bottle; 51. positioning groove; 52. bottle stopper; 53. cotton core; 6. light-emitting assembly; 7. plug; 8. rotating member; 81. rotating stop block; 82. rotating abutting block; 9. rotating position groove; 91. horizontal position groove; 92. inclined position groove; and 93. vertical position groove.

### DETAILED DESCRIPTIONS OF THE EMBODIMENTS

The technical solutions in the examples of the present disclosure will be clearly and completely described below with reference to the accompanying drawings in the examples of the present disclosure.

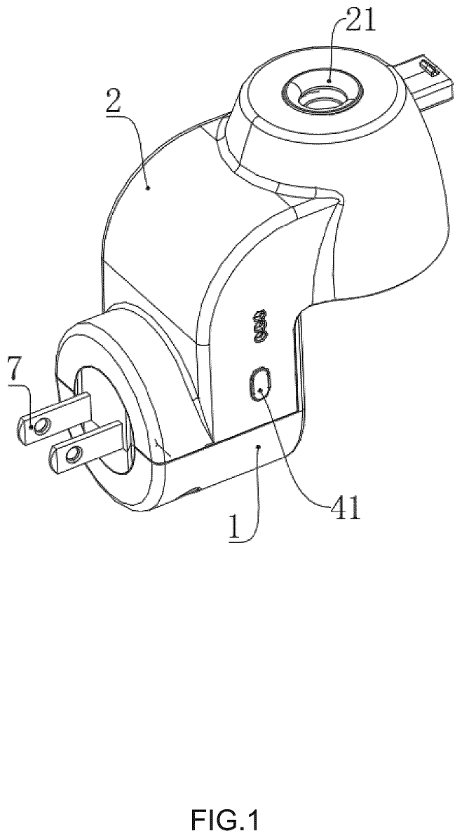

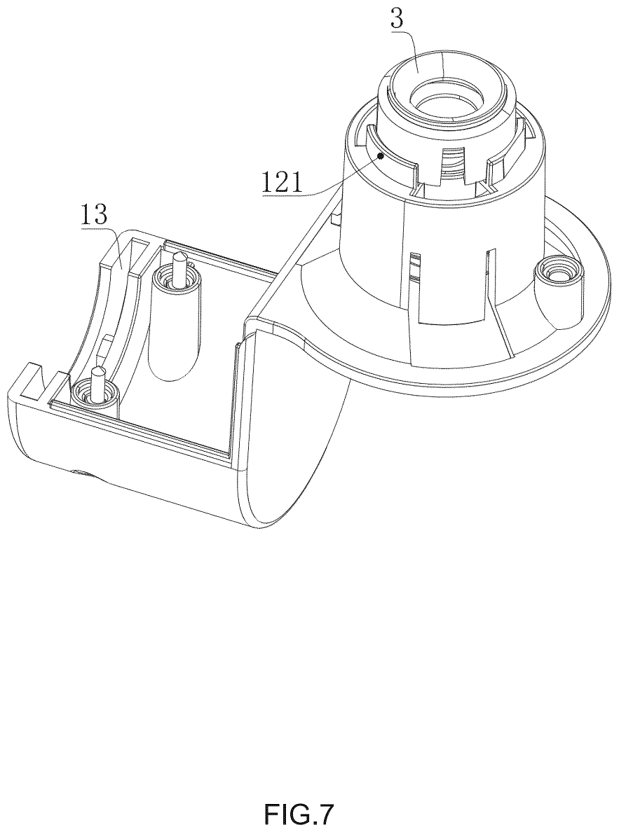

### Refer to FIGS. 1-9.

As illustrated in FIGS. 1-6, an aroma diffuser of the present disclosure includes a housing and an atomizing device 3. The housing is provided with an object mounting site 11, an air outlet 21, and an atomizing mounting site 12. The atomizing device 3 includes an atomizing shell 31, an atomizing sheet 32, and an atomizing elastic member 33. At least one atomizing hole 34 is formed in the atomizing sheet 32, the atomizing elastic member 33 and the atomizing sheet 32 are mounted in the atomizing shell 31, and the atomizing shell 31 is provided with an atomizing snap-fit site corresponding to the atomizing mounting site 12. It should be noted that the object mounting site 11 can be configured for fixing an aroma bottle 5. The atomizing device 3 is arranged between the object mounting site 11 and the air outlet 21. During assembly, the atomizing sheet 32, the atomizing elastic member 33, and the atomizing shell 31 are assembled outside the housing to form the atomizing device 3. Then the entire atomizing device 3 is inserted into the housing, and the atomizing snap-fit site is snap-fitted on the atomizing mounting site 12 to achieve mounting and fixation of the atomizing device 3 to the housing. A simple structure and the assembly outside the housing avoid positioning of the atomizing sheet 32 and the atomizing elastic member 33 with the housing, thereby effectively improving assembly efficiency and reducing assembly time and costs. When in use, the aroma bottle 5 is placed at the object mounting site 11, and the atomizing sheet 32 corresponds to an opening of the aroma bottle 5. When the atomizing elastic member 33 and the atomizing sheet 32 vibrate at a high frequency at the opening of the aroma bottle 5, liquid in the aroma bottle 5, after passing through the atomizing hole 34, is mixed with a water mist formed by air and then discharged from the air outlet 21, to achieve uniform atomization and diffusion of diffuser liquid.

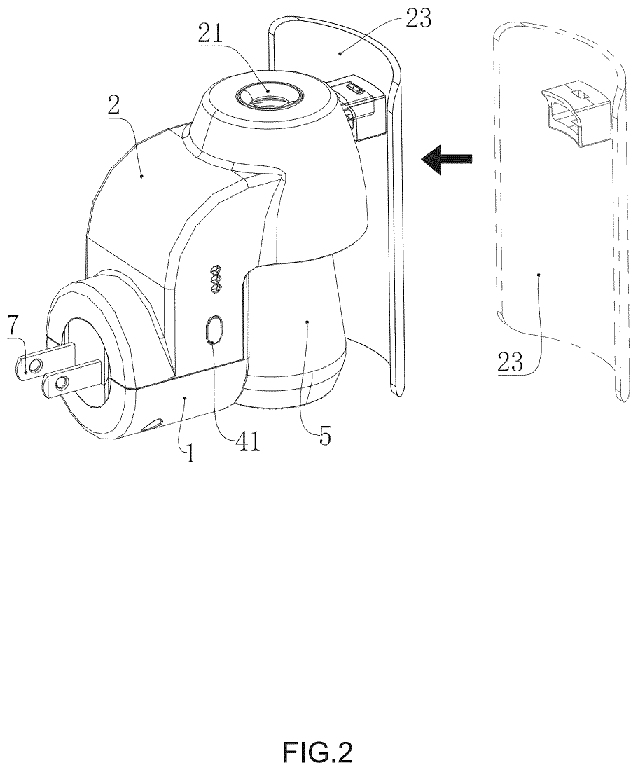

As illustrated in FIGS. 2-7, the housing includes an upper housing 2 and a lower housing 1, where the object mounting site 11 and the atomizing mounting site 12 are arranged on the lower housing 1, and the air outlet 21 is arranged on the upper housing 2. The atomizing mounting site 12 is located at an end of the object mounting site 11, and the atomizing mounting site 12 includes vertical mounting plates 121, horizontal mounting plates 122, and a bottleneck limiting block 123. An outflow hole 124 is formed in the bottleneck limiting block 123, and a recess 125 is formed in each of the horizontal mounting plates 122. In this example, a mounting cavity that is structurally compatible with the atomizing shell 31 is formed between the vertical mounting plates 121 (three ones) and the horizontal mounting plates 122 (three ones). The atomizing snap-fit site includes snap latches 35. The atomizing shell 31 of the atomizing device 3 is aligned with the mounting cavity, and the snap latches 35 are aligned with and pressed into the recesses 125. In this case, the snap latches 35 are snap-fitted into the recesses 125, such that the atomizing device 3 is fixed at a position of the mounting cavity in a snap-fitting manner. A position of the atomizing sheet 32 corresponds to the outflow hole 124 of the bottleneck limiting block 123. When disassembly, replacement or maintenance is needed, a user only needs to pull out the atomizing device 3 in a direction away from the atomizing mounting site 12 to disengage the snap latches 35 from the recesses 125. Due to simple disassembly and assembly steps, each user can easily complete the assembly and replacement.

Specifically, to achieve both an illumination effect and a certain decorative effect, the atomizing shell 31 can be made of a translucent material, and a light-emitting assembly 6 is arranged in the upper housing 2. In this example, the light-emitting assembly 6 is an LED lamp panel, and a light-emitting end of the LED lamp panel faces a top portion of the air outlet 21 to create the illumination effect.

Specifically, a detachable decorative board 23 is located outside the upper housing 2, and the decorative board 23 can be provided with various patterns. According to actual needs of users, the decorative board 23 can be assembled together with the upper housing 2 to make the aroma diffuser look more beautiful, and can also be removed from the upper housing 2, thereby facilitating flexible use.

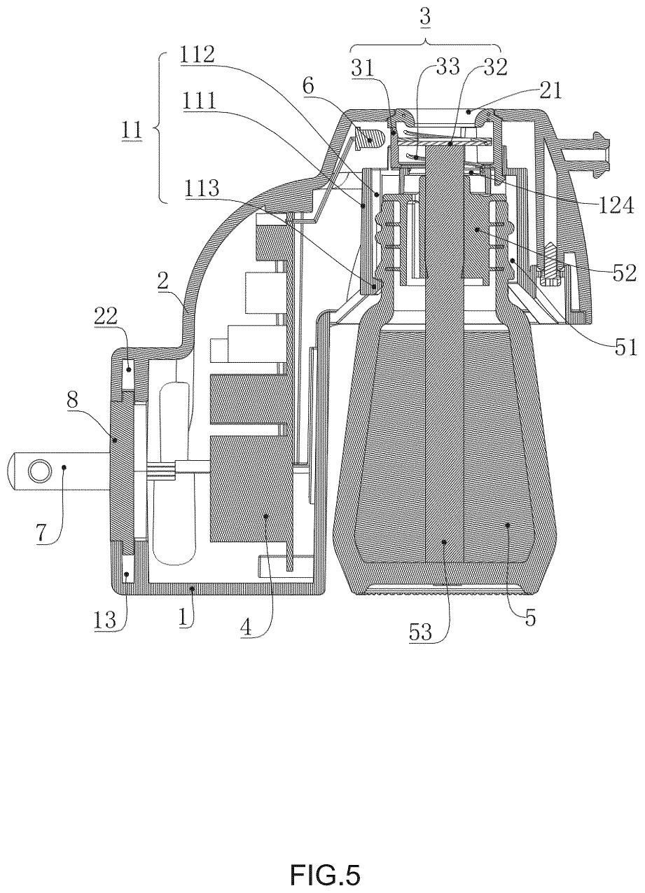

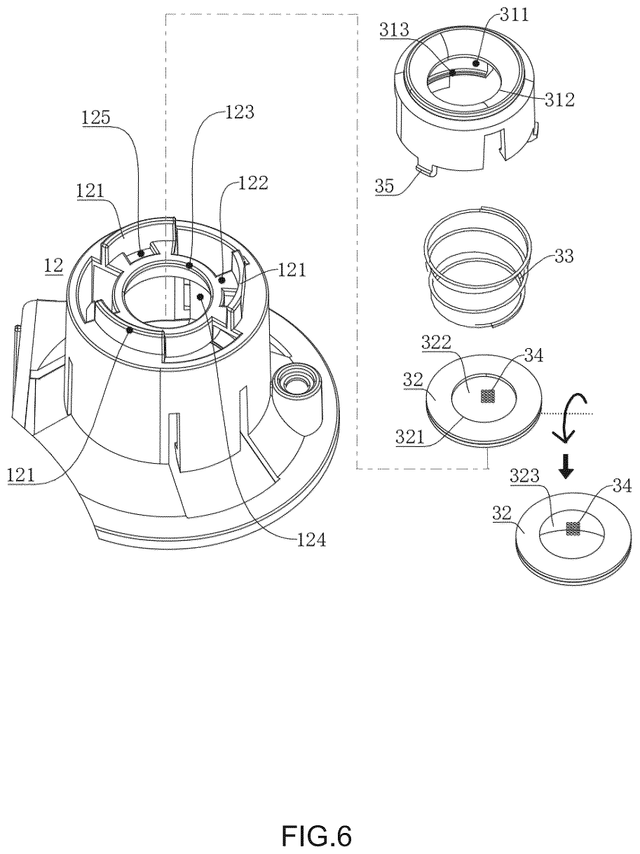

As illustrated in FIGS. 5-6, the atomizing shell 31 is provided with an atomizing chamber 311, and the atomizing sheet 32 and the atomizing elastic member 33 are placed inside the atomizing chamber 311. One end of the atomizing chamber 311 is provided with a mist discharge hole 312, and the other end thereof is provided with a stop block 313, where the stop block 313 abuts against and limits the atomizing sheet 32 in the atomizing chamber 311, to prevent the atomizing sheet 32 and the atomizing elastic member 33 from detaching from the atomizing chamber 311, thereby avoiding failures of atomizing.

Specifically, the position of the atomizing sheet 32 corresponds to the outflow hole 124 of the bottleneck limiting block 123. The lower housing 1 is provided with the control circuit device 4, and the atomizing sheet 32 is electrically connected to the control circuit device 4. The atomizing sheet 32 is provided with an atomizing site 321, and the atomizing hole 34 is formed in the atomizing site 321. The atomizing site 321 includes a concave surface 322 and a convex surface 323, where the concave surface 322 is arranged on a side of the outflow hole 124, and the convex surface 323 is arranged on a side of the mist discharge hole 312. Under control of the control circuit device 4, high-frequency vibration caused by the atomizing sheet 32 results in that essential oil in the aroma bottle 5 is decomposed into nano-scale cold mist with a diameter of 0.1-5 microns at a position of the concave surface 322, and then the nano-scale cold mist is diffused in surrounding air gradually through the convex surface 323, such that the air is filled with fragrance. Arrangement of the concave surface 322 and the convex surface 323 enhances the atomizing effect, broadens a dispersion range, and improves atomizing efficiency.

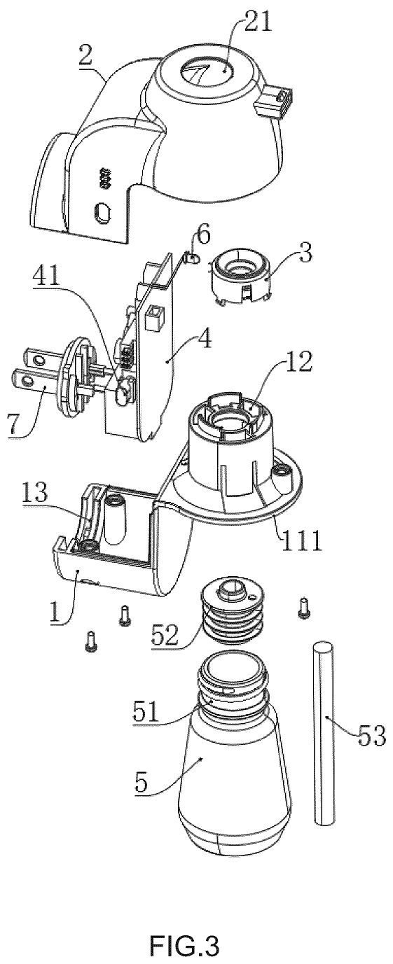

As illustrated in FIG. 3, the lower housing 1 is provided with a control switch 41, and the control switch 41 is electrically connected to a control circuit device 4. In this example, the control switch 41 has three gear positions: fast, medium, and slow. Through adjustment of the control switch 41, the atomizing sheet 32 can vibrate at a fast, medium or slow speed, to achieve a desired atomizing effect.

Specifically, the aroma bottle 5 includes a bottle stopper 52 and a cotton core 53. An upper end of the cotton core 53 corresponds to a position of the outflow hole 124 and is in contact with the concave surface 322 of the atomizing sheet 32, and a lower end of the cotton core 53 passes through the bottle stopper 52 and enters an interior of the aroma bottle 5, and makes contact with a bottom of the aroma bottle 5, such that the essential oil inside the aroma bottle 5 can be continuously supplied upward through an adsorption action of the cotton core 53. The atomizing sheet 32 and the concave surface 322, in the high-frequency vibration state, press the cotton core 53, and water molecules and dissolved essential oil inside the cotton core 53 are decomposed into the nano-scale cold mist with a diameter of 0.1-5 microns at a position of the concave surface 322, and the nano-scale cold mist is then diffused into the surrounding air through the convex surface 323.

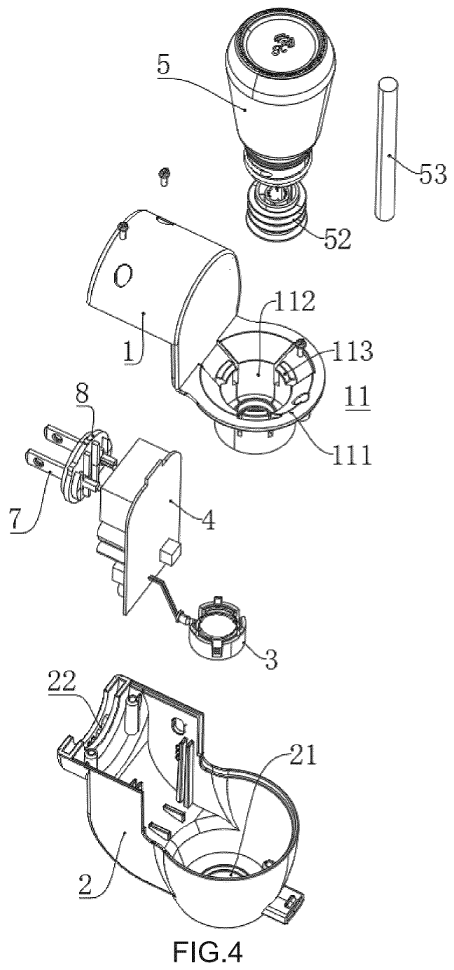

As illustrated in FIGS. 4-5, the object mounting site 11 includes a mounting bracket 111, the mounting bracket 111 is recessed downward along its upper end to form an avoidance channel 112 that matches the aroma bottle 5 in a shape. The aroma bottle 5 is detachably mounted in the avoidance channel 112, the mounting bracket 111 is provided with a positioning latch 113 at an upper end of the avoidance channel 112, and the aroma bottle 5 is provided with a positioning groove 51 that fits the positioning latch 113. When necessary to add the essential oil, the user can directly remove the aroma bottle 5 from the avoidance channel 112 and reinstall the aroma bottle 5 in the avoidance channel 112 after adding the essential oil. Cooperation between the positioning latch 113 and the positioning groove 51 facilitates positioning and fixing of the aroma bottle 5, and prevents the aroma bottle 5 from being loosened.

As illustrated in FIGS. 3-5, a side of the control circuit device 4 is electrically connected to a plug 7, a rotating member 8 is provided on a side of the plug 7, and the plug 7 is mounted on the rotating member 8. The lower housing 1 is provided with an upper rotating mounting arc groove 22 that corresponds to the rotating member 8, and the upper housing 2 is provided with a lower rotating mounting arc groove 13 that corresponds to the rotating member 8. The rotating member 8 is simultaneously snap-fitted into both the lower rotating mounting arc groove 13 and the upper rotating mounting arc groove 22 to achieve fixation between the upper housing 2 and the lower housing 1, so as to fix the plug 7 on both the upper housing 2 and the lower housing 1.

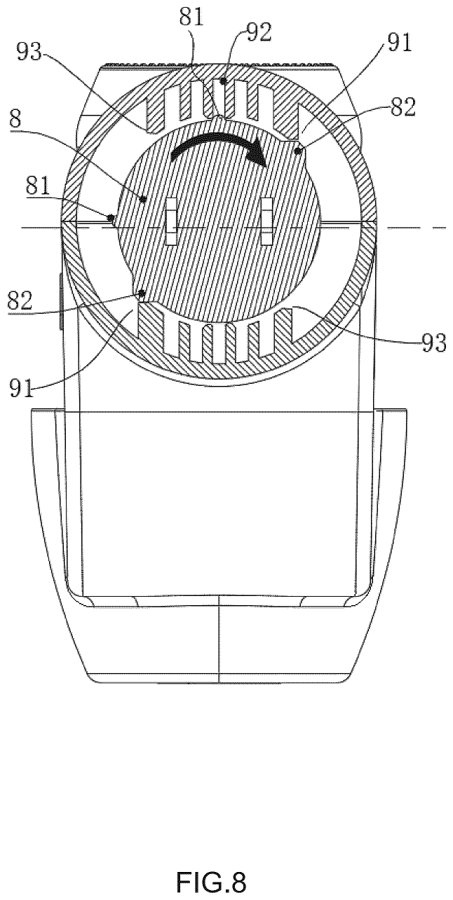

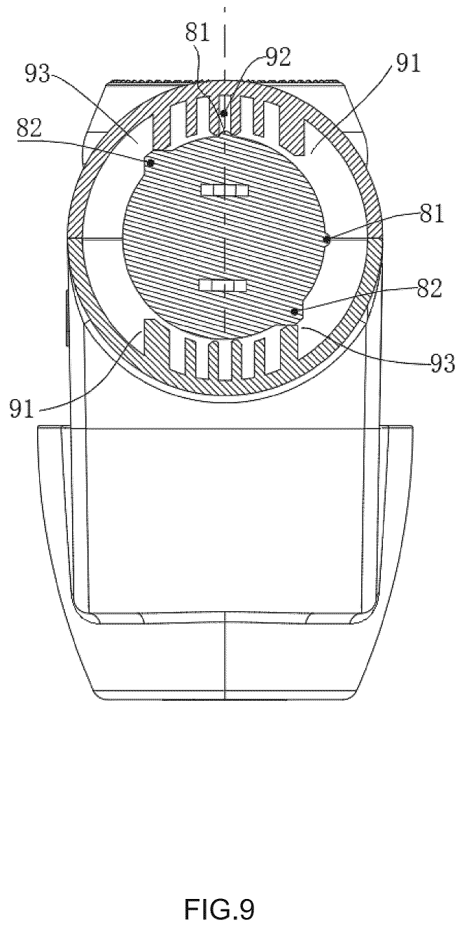

As illustrated in FIGS. 8-9, the lower rotating mounting arc groove 13 and/or the upper rotating mounting arc groove 22 is provided with a rotating position groove 9. The rotating member 8 is provided with at least one rotating stop block 81 and one rotating abutting block 82. The rotating position groove 9 includes a horizontal position groove 91, an inclined position groove 92, and a vertical position groove 93. The rotating stop block 81 is snap-fitted into the inclined position groove 92. When two jacks of a socket are horizontally arranged, the rotating member 8 can be rotated between the lower housing 1 and the upper housing 2 such that the rotating abutting block 82 abuts against the horizontal position groove 91, thereby ensuring that the plug 7 and the two jacks are simultaneously aligned on a same horizontal line for easy insertion. When the two jacks of the socket are vertically arranged, the rotating member 8 can be rotated between the lower housing 1 and the upper housing 2 such that the rotating abutting block 82 abuts against the vertical position groove 93, thereby ensuring that the plug 7 and the two jacks are simultaneously aligned on a same vertical line for easy insertion. The arrangement of the rotating position groove 9 ensures that the plug 7 adapts to different socket arrangement modes, thereby enhancing flexibility of use and effectively optimizing user experience.

For those skilled in the art, it is apparent that the present disclosure is not limited to the details of the above-mentioned exemplary examples, and the present disclosure can be implemented in other specific forms without departing from the spirit or basic features of the present disclosure. Therefore, no matter from which point of view, the examples should all be regarded as exemplary and non-limiting. The scope of the present disclosure is defined by the appended claims rather than the above-mentioned description, and therefore it is intended that all changes which fall within the meaning and scope of equivalency of the claims are embraced in the present disclosure. Any reference numeral in the claims should not be construed as limiting the related claims.

In addition, it should be understood that although the present description is described according to the embodiments, not each embodiment only contains an independent technical solution, and the specification manner of the description is only for clarity. Those skilled in the art should take the description as a whole, and the technical solutions in the examples can also be appropriately combined to form other embodiments that can be understood by those skilled in the art.

What is claimed is:

1. An aroma diffuser, comprising a housing and an atomizing device, wherein the housing is provided with an atomizing mounting site, the atomizing device comprises an atomizing shell, an atomizing sheet and an atomizing elastic member, at least one atomizing hole is formed in the atomizing sheet, and the atomizing shell is provided with an atomizing snap-fit site corresponding to the atomizing mounting site.

2. The aroma diffuser according to claim 1, wherein the housing is provided with an object mounting site and an air outlet, the atomizing shell is provided with an atomizing chamber, one end of the atomizing chamber is provided with a mist discharge hole, and the other end thereof is provided with a stop block.

3. The aroma diffuser according to claim 2, wherein the housing comprises an upper housing and a lower housing, the atomizing mounting site comprises vertical mounting plates, horizontal mounting plates, and a bottleneck limiting block, and an outflow hole is formed in the bottleneck limiting block.

4. The aroma diffuser according to claim 3, wherein a recess is formed in each of the horizontal mounting plates, a mounting cavity is formed between the vertical mounting plates and the horizontal mounting plates, and the atomizing snap-fit site comprises snap latches.

5. The aroma diffuser according to claim 4, wherein lower housing is provided with a control circuit device, the atomizing sheet is electrically connected to the control circuit device, the atomizing sheet is provided with an atomizing site, the atomizing hole is formed in the atomizing site, and the atomizing site comprises a concave surface and a convex surface, wherein the concave surface is arranged on a side of the outflow hole, and the convex surface is arranged on a side of the mist discharge hole.

6. The aroma diffuser according to claim 5, wherein the object mounting site comprises a mounting bracket, the mounting bracket is recessed downward along its upper end to form an avoidance channel that matches an aroma bottle in a shape, the mounting bracket is provided with a positioning latch at an upper end of the avoidance channel, and the aroma bottle is provided with a positioning groove that fits the positioning latch.

7. The aroma diffuser according to claim 6, wherein the aroma bottle comprises a bottle stopper and a cotton core, an upper end of the cotton core corresponds to a position of the outflow hole, and a lower end of the cotton core passes through the bottle stopper and enters an interior of the aroma bottle, making contact with a bottom of the aroma bottle.

8. The aroma diffuser according to claim 7, wherein a light-emitting assembly is arranged in the upper housing, and a detachable decorative board is located outside the upper housing.

9. The aroma diffuser according to claim 8, wherein a side of the control circuit device is electrically connected to a plug, a rotating member is provided on a side of the plug, the lower housing is provided with an upper rotating mounting arc groove, and the upper housing is provided with a lower rotating mounting arc groove.

10. The aroma diffuser according to claim 9, wherein the lower rotating mounting arc groove and/or the upper rotating mounting arc groove is provided with a rotating position groove, the rotating member is provided with at least one rotating stop block and one rotating abutting block, and the rotating position groove comprises a horizontal position groove, an inclined position groove, and a vertical position groove.
